(19) United States
(12) Patent Application Publication
(10) Pub. No.: US 2019/0231265 A1
(43) Pub. Date: Aug. 1, 2019

(54) STRETCHABLE IONO-ELASTOMERS WITH MECHANO-ELECTRICAL RESPONSE, DEVICES INCORPORATING IONO-ELASTOMERS, AND METHODS OF MAKING THEREOF

(71) Applicants: Carlos R. López-Barrón, Houston, TX (US); Ru Chen, Brooklyn, NY (US); Norman J. Wagner, Newark, DE (US)

(72) Inventors: Carlos R. López-Barrón, Houston, TX (US); Ru Chen, Brooklyn, NY (US); Norman J. Wagner, Newark, DE (US)

(73) Assignee: University of Delaware, Newark, DE (US)

(21) Appl. No.: 16/332,297

(22) PCT Filed: Apr. 7, 2017
(86) PCT No.: PCT/US17/26621

§ 371 Date: Mar. 11, 2019

Related U.S. Application Data
(60) Provisional application No. 62/393,133, filed on Sep. 12, 2016.

(51) Int. Cl. A61B 5/00; A61B 5/11; C08L 53/00

(52) U.S. Cl. CPC A61B 5/6806; A61B 5/1126; A61B 5/1107; A61B 5/0002; H01M 10/425; A61B 5/6833; A61B 2562/0261; A61B 2560/0214; C08L 2203/02; C08L 53/005

(57) ABSTRACT

Conductive, elastic ionic polymers with high tensile strength (iono-elastomers) resulting from amphiphilic polymers capable of self-assembly into micellar hierarchical gel-like structures in an electrically conductive liquid are provided. The micellar hierarchical gel-like structures engage in micellar corona cross-linking to form the iono-elastomers. The iono-elastomer exhibits high stretchability and displays increased electrical conductivity upon mechanical stretching. A method for producing iono-elastomers includes mixing amphiphilic polymers in a conductive liquid to form micellar hierarchical gel-like structures, and cross-linking the micellar coronas. Devices incorporating iono-elastomers and methods for creating such devices are also provided. The devices incorporating the iono-elastomers may be wearable.

### CROSS-REFERENCE TO RELATED APPLICATIONS

This application claims the benefit of priority of U.S. Provisional Application No. 62/393,133, filed Sep. 12, 2016, which application is incorporated by reference herein, in its entirety and for all purposes.

### STATEMENT OF GOVERNMENT INTEREST

This invention was made with Government support under Grant No. 1247394, awarded by the National Science Foundation Graduate Research Fellowship, as well as Grant Nos. 70NANB12H239 and 70NANB15H260, awarded by the National Institute of Standards and Technology, U.S. Department of Commerce. The Government has certain rights in the invention.

### FIELD OF THE INVENTION

This invention relates to the field of electrically conductive, elastic polymeric gels and elastomers and, more particularly, devices and sensors made with conductive, elastic polymeric gels and elastomers and methods of making thereof.

### BACKGROUND OF THE INVENTION

Structural materials with high conductivity and high stretchability are becoming an important area of research due to emerging technologies involving wearable electronics. Wearable electronics and sensors to be incorporated into clothing, uniforms, and sporting equipment require flexible, robust soft materials with tunable conductivity and high extensibility—so called “stretchable electronics”. These technologies require conductive stretchable materials that sustain loads without losing their conductive characteristics upon stretching. Applications of these materials include stretchable batteries, wearable sensors, and integrated circuits.

Current fabrication techniques for these materials require the use of hybrid technologies that mix metals, polymers and conductive materials, to tie the system together electronically. Some of the most commonly explored strategies include composite materials with “wavy” or fractal designs, micro- or nano-structured metal embedded in elastomeric matrices, biphasic solid-liquid metal thin films embedded in elastomeric substrates, composite films of carbon nanotubes or graphene, and metals and polymeric materials doped with dispersions of carbon nanotubes in ionic liquids (bucky gels) which are subsequently coated with elastomeric substrates. Methods to manufacture these materials involve sophisticated and complex integration of elastomeric substrates with micro- or nano-structured organic or inorganic electronic materials via multi-step and often costly processes. It follows that a simplified manufacturing process of stretchable and conductive materials would be of great value to scale-up these new stretchable electronic technologies.

An alternative strategy to produce stretchable conductors is to use ionic liquids (ILs) as the conductive media. Previous studies have reported in-situ polymerization of vinyl monomers in ILs, which results in flexible conductive gels (ion-gels). The synthesis of a poly(styrene-b-ethylene oxide-b-styrene) triblock copolymer, in which 25% of the styrene units have a pendant azide functionality, has also been reported. Self-assembly of this copolymer in IL, followed by cross-linking of the azide groups produces conductive solid ion gels with strain-to-break values of—350%. However, because these materials are not hierarchically ordered, their tensile properties are inadequate to support use in, for example, wearable electronics.

Another strategy is to employ elastomer sheets coated with bucky gels (carbon nanotube/ionic liquid composites) produce highly conductive flexible materials. However, the stretchability of these materials is limited to strain-to-break values <150%, and their production is multi step. Additionally, due to the strong optical absorption in carbon nanotubes, bucky gels are opaque, which could be detrimental for some applications.

Therefore, a significant challenge for the emerging technologies involving wearable electronics is to design materials with high and reproducible stretchability, high electrical conductivities, and resistance to high loads, while keeping fabrication processes simple to accommodate economic considerations. Complex multistep fabrication procedures are discouraged, as they drive up costs.

Muscle injuries are extremely common amongst athletes, emergency personnel, and the military, and could be detrimental to the careers of such individuals if recovery is not properly carried out. Moreover, research has revealed that muscle damage is not a function of muscle force, but of active muscle strain. However, current muscular tracking devices can only monitor entire body performance via metrics, such as heart rate, speed and distance. There is no wearable tracking device that can monitor local muscle strain under workload. It would therefore be useful to have soft, elastic materials capable of incorporation into wearable devices and/or garments to measure and track an individual's potential for muscle injury, or recovery therefrom.

### SUMMARY OF THE INVENTION

Aspects of the invention relate to ionically conductive, elastic polymers (iono-elastomers), as well as sensors incorporating iono-elastomers and processes for producing iono-elastomers and sensors incorporating iono-elastomers.

In accordance with one aspect, the invention provides a conductive, stretchable iono-elastomer. The iono-elastomer is composed of an amphiphilic polymer and/or block co-polymer capable of hierarchical self-assembly into organized micellar structures in an electrically conductive liquid solvent. The hierarchically organized micellar structures engage in micellar corona cross-linking and bridge crosslinking by soluble polymers. Such crosslinked, hierarchically organized iono-elastomers exhibit high stretchability and unexpectedly display increased electrical conductivity upon mechanical stretching.

In accordance with another aspect, the invention provides a method for producing iono-elastomers. The method is a simple two-step process, which includes mixing end-functionalized amphiphilic polymers in an electrically conductive liquid to form hierarchical micellar structures, and cross-linking the micellar coronas.

In accordance with yet another aspect, the invention provides a device incorporating one or more iono-elastomers. The devices may be sensors, which sensors may be incorporated into stretchable electronic technologies, including wearable technologies.

It is to be understood that both the foregoing general description and the following detailed description are exemplary, but are not restrictive, of the invention.

### BRIEF DESCRIPTION OF THE DRAWINGS

The invention is best understood from the following detailed description when read in connection with the accompanying drawings, with like elements having the same reference numerals. When a plurality of similar elements are present, a single reference numeral may be assigned to the plurality of similar elements with a small letter designation referring to specific elements. When referring to the elements collectively or to a non-specific one or more of the elements, the small letter designation may be dropped. This emphasizes that according to common practice, the various features of the drawings are not drawn to scale unless otherwise indicated. On the contrary, the dimensions of the various features may be expanded or reduced for clarity. Included in the drawings are the following figures:

FIG. 1 is a schematic illustration of the self-assembly of F127-DA in dEAN and a subsequent photo-cross-linking reaction, according to principles of the present invention. The inset of FIG. 1 depicts small-angle neutron scattering profiles for a 24 wt % F127-DA/dEAN solution before and after cross-linking at 40° C., in which solid lines are best fits to a paracrystalline FCC lattice model.

FIG. 2a is a graphical depiction of a stress-strain curve for a cross-linked 24 wt % F127-DA/dEAN iono-elastomer measured in a Sentmanat extensional rheometer (SER) with a Henckey strain rate of 0.01 s⁻¹, according to principles of the present invention.

FIG. 2b is a graphical depiction of a stress curve for a cross-linked 24 wt % F127-DA/dEAN iono-elastomer measured in a SER with a Henckey strain rate of 0.01 s⁻¹ as a function of time showing loading to Hencky strains of 0.032, 0.32, 2, and 4, followed by relaxation for one hour, according to principles of the present invention.

FIG. 3 is a schematic illustration of a postulated microstructure obtained after micelle corona cross-linking of an iono-elastomer showing adjacent and bridged inter-micellar cross-links as well as intra-micellular cross-links, along with an illustration of two perpendicular views of a layered hexagonal close-packed (HCP) structure observed by neutron scattering during stretching of an iono-elastomer, according to principles of the present invention.

FIG. 4a is graphical depiction of a hysteresis test performed on a cross-linked 24 wt % F127-DA/dEAN iono-elastomer in the SER showing consecutive loading-unloading cycles, with Hencky strain rate of 0.01 s⁻¹ to a strain value of 10%, according to principles of the present invention.

FIG. 4b is graphical depiction of a hysteresis test performed on a cross-linked 24 wt % F127-DA/dEAN iono-elastomer in the SER showing consecutive loading-unloading cycles, with Hencky strain rate of 0.01 s⁻¹ to a strain value of 38%, according to principles of the present invention.

FIG. 4c is graphical depiction of a hysteresis test performed on a cross-linked 24 wt % F127-DA/dEAN iono-elastomer in the SER showing consecutive loading-unloading cycles, with Hencky strain rate of 0.01 s⁻¹ to a strain value of 505%, according to principles of the present invention.

FIG. 4d is graphical depiction of a hysteresis test performed on a cross-linked 24 wt % F127-DA/dEAN iono-elastomer in the SER showing consecutive loading-unloading cycles with subsequent increase in strain, resulting in significant increase in hysteresis, according to principles of the present invention.

FIG. 4e is graphical depiction of tensile set and dissipated energy of a cross-linked 24 wt % F127-DA/dEAN iono-elastomer in the SER as a function of subsequent loading-unloading cycles, in which squares, circles, triangles, and diamonds correspond to data in FIGS. 4a, 4b, 4c, and 4d, respectively, according to principles of the present invention.

FIG. 5a is a photograph depicting LED lights connected to a battery via a film of cross-linked 24 wt % F127-DA/dEAN iono-elastomer, displaying the iono-elastomer's conductivity, according to principles of the present invention.

FIG. 5b is a photograph depicting an experimental set up for simultaneous measurements of uniaxial stress (with a rheometer) and electrical resistance with the Ohmmeter of cross-linked 24 wt % F127-DA/dEAN iono-elastomer, according to principles of the present invention.

FIG. 5c is a graphical depiction of normalized electrical resistance and stress as a function of time corresponding to the hysteresis test performed on a cross-linked 24 wt % F127-DA/dEAN iono-elastomer in the SER showing consecutive loading-unloading cycles of FIG. 4a, according to principles of the present invention.

FIG. 5d is a graphical depiction of normalized electrical resistance and stress as a function of time corresponding to the hysteresis test performed on a cross-linked 24 wt % F127-DA/dEAN iono-elastomer in the SER showing consecutive loading-unloading cycles of FIG. 4b, according to principles of the present invention.

FIG. 5e is a graphical depiction of normalized electrical resistance and stress as a function of time corresponding to the hysteresis test performed on a cross-linked 24 wt % F127-DA/dEAN iono-elastomer in the SER showing consecutive loading-unloading cycles of FIG. 4c, according to principles of the present invention.

FIG. 5f is a graphical depiction of normalized electrical resistance and stress as a function of time corresponding to the hysteresis test performed on a cross-linked 24 wt % F127-DA/dEAN iono-elastomer in the SER showing consecutive loading-unloading cycles of FIG. 4d, according to principles of the present invention.

FIG. 5g is a graphical depiction of normalized electrical resistance and stress as a function of elongation strain performed on a cross-linked 24 wt % F127-DA/dEAN iono-elastomer, according to principles of the present invention.

FIG. 6a is a photograph depicting a Linkam tensile stage with an iono-elastomer sample undergoing stretching, according to principles of the present invention.

FIG. 6b is a graphical illustration of a stress-strain curve measured on an iono-elastomer sample in the Linkam tensile stage, and 2D small-angle neutron scattering (SANS) measurement profiles measured at the strain values indicated by the arrows, including SANS profiles measured before and after loading, according to principles of the present invention.

FIG. 6c is a graphical illustration of annularly-averaged SANS intensity for an iono-elastomer sample as a function of azimuthal angle, with the annular sector used for the intensity averaging centered on the mean scattering peak (0.025 Å⁻¹≤q≤0.06 Å⁻¹), according to principles of the present invention.

FIG. 6d is a graphical representation of azimuthally-averaged SANS intensity as a function of scattering vector, q, measured before and after loading, as well as at a Hencky strain of 1.62, according to principles of the present invention.

FIG. 7a is a schematic illustration of a device incorporating the iono-elastomers as a transparent sensor patch composed of three layered structures, according to principles of the present invention.

FIG. 7b is a schematic illustration of a device patch incorporating the iono-elastomers and having a wireless communication and transmission system, according to principles of the present invention.

FIG. 8 is a photograph depicting a glove device incorporating the iono-elastomers as a muscle strain measuring device, according to principles of the present invention.

FIG. 9 is a schematic illustration of preparation of F127-DA/dEAN solution and crosslinking to form an iono-elastomer, according to principles of the present invention.

### DETAILED DESCRIPTION OF THE INVENTION

Aspects of the invention are directed to iono-elastomers, as well as devices incorporating iono-elastomers and processes for producing iono-elastomers and devices incorporating iono-elastomers.

### Iono-Elastomers

The inventors have recognized that it would be useful to provide stretchable materials with tunable conductivity applicable for stretchable electronic technologies. The inventors have also recognized a need for facile, inexpensive synthesis of transparent, conductive soft elastomers via self-assembly of an amphiphilic polymer or block copolymer in a conductive liquid into hierarchically organized gel-like materials, followed by crosslinking. The inventors have further recognized that it would be useful to use inexpensive, commercially available raw materials to produce iono-elastomers. These elastomers have high conductivity, remarkable tensile properties, and mechano-electrical response. The inventors have recognized that the mechano-electrical response property can be used for design of motion sensors and devices for wearable electronics.

The inventors have demonstrated a simplified two-step manufacturing process to create stretchable materials with tunable conductivity applicable for stretchable electronic technologies by hierarchical self-assembly of concentrated solutions of end-functionalized, commercially available, inexpensive amphiphilic polymers and copolymers into micelles in a conductive liquid, followed by chemical crosslinking. In addition to the amphiphilic polymers, solvophilic and/or soluble polymers/copolymers (e.g., PEO diacrylate) may be added to the concentrated solutions to tune crosslinking and aid in forming bridged crosslinks.

In certain embodiments of the invention, the end group-functionalized amphiphilic polymer comprises at least one polyethylene oxide (PEO) block (e.g., one, two, three or more PEO blocks). Such PEO blocks can function as the solvolytic portion(s) of the amphiphilic polymer. The PEO block(s) may have a number average molecular weight of from about 500 Daltons (approximately 11 repeating units of ethylene oxide) to about 10,000 Daltons (approximately 230 repeating units of ethylene oxide), for example. According to other embodiments, the solvophobic portion(s) of the amphiphilic polymer may be provided in the form of one or more polypropylene oxide (PPO) blocks. The PPO block(s) may have, for example, a number average molecular weight of from about 500 Daltons (approximately 9 repeating units of propylene oxide) to about 10,000 Daltons (approximately 170 repeating units of propylene oxide). End group-functionalized amphiphilic polymers useful in the present invention may have, for example, number average molecular weights of from about 1000 Daltons to about 20,000 Daltons.

The amphiphilic polymer may be linear or branched. In certain embodiments, the amphiphilic polymer used to prepare the iono-elastomer is difunctional (i.e., the amphiphilic polymer contains two functionalized end groups capable of participating in a crosslinking reaction). However, it is also possible for the amphiphilic polymer to contain more than two functionalized end groups.

Suitable end-functionalizations include any chemical group capable of undergoing chemical crosslinking, including but not limited to, acrylate, methacrylate, thiolene, carboxyl, amine, sulfhydryl, aldehyde, azide, cyanate, esters, carbodiimide, vinyl groups, acrylamide, norborane, epoxy, and isocyanate. Chemical crosslinking of the functionalized end groups of the amphiphilic polymer/copolymers may be achieved by any suitable means, depending on the nature of the functionalized end groups. For example, the functional end groups may be capable of undergoing crosslinking reactions when exposed to a suitable source of radiation (e.g., ultraviolet light of electron beam radiation). A crosslinking agent capable of reacting with the functionalized end groups could also be used.

Suitable concentration ranges of polymer/copolymer required to produce an iono-elastomer vary depending on the combination of electrically conductive liquid solvent and the type of polymer/copolymer used, but the polymer/copolymer is preferably present in a concentration of between 5 wt % and 60 wt %. More preferably, the polymer/copolymer is present in a concentration of between 10 wt % and 45 wt %. Even more preferably, the polymer/copolymer is present in a concentration of between 15 wt % and 35 wt %.

The resulting iono-elastomer materials exhibit transparency, an unprecedented combination of high stretchability (elongation at break is 3000% and tensile strength at break is 200 MPa), tunable ionic conductivity and mechano-electrical response. The stretchability of these iono-elastomers may be up to approximately 30 times higher than other reported elastomers.

The inventors have also surprisingly and unexpectedly found that the electrical resistance of these iono-elastomers decreases with mechanical extension, which is a unique and non-trivial material response. Such a property is counterintuitive, as electrical resistance is expected to increase with strain and distance. Without being bound by any particular theory, the inventors postulate that this property is a result of the microstructural rearrangement of the micelles when the iono-elastomer is placed under uniaxial deformation. Evidence for such a rearrangement is supported by neutron scattering.

The favorable properties of the iono-elastomers as described above make them uniquely suited for use in stretchable electronics, including wearable electronics. For example, the iono-elastomers may be incorporated into a transparent stretchable muscle strain sensor, which measures the local displacement of muscle under workload, for pro-athletes in use for sports injury prevention, performance analysis and recovery tracking. Such sensors may be form factor free devices and can be directly attached to skin. Incorporating the iono-elastomers may result in sensors having lighter weights and decreased raw material costs in comparison to other sensors used for similar purposes.

The iono-elastomers could be used in any situation requiring durable, stretchable, conductive elastic materials. Athletic clothing, military applications, and police, firefighter, and industrial uniforms could benefit from integration of these highly extensible, flexible conductive materials for use as sensors and electrical connectors for communication and other added functionalities. Aside from these benefits, the iono-elastomers may be made of environmentally friendly raw materials which will help reduce production waste. Additionally, the raw material cost for the iono-elastomers is low.

As used herein, “iono-elastomers” refer collectively to a group of highly stretchable, electrically conductive elastomeric materials capable of mechano-electrical response. The iono-elastomers are formed from hierarchical structures comprising micelles which are self-assembled from amphiphilic polymers/copolymers in conductive liquid and engage in cross-linking to generate elastomeric ion gels.

As used herein, “solvophilic” refers to the characteristic of a non-crosslinked polymer/copolymer subunit molecule or portion of the polymer/copolymer subunit molecule as understood herein having an affinity for an electrically conductive liquid solvent as understood herein, such that the polymer subunit molecule is capable of dissolving in the electrically conductive liquid solvent at a concentration of at least 0.01 wt % at a range preferably between 12° C. and 100° C.

As used herein, “amphiphilic” refers to the characteristic of a non-crosslinked polymer subunit molecule as understood herein having a portion that is solvophilic and a portion that is solvophobic (lacking an affinity for an electrically conductive liquid such that the portion resists dissolution in the electrically conductive liquid solvent).

As used herein, “hierarchical” refers to the characteristic of a plurality of amphiphilic polymers to self-assemble into micelles within electrically conductive liquid, which micelles self-organize into a further structure (e.g., crystalline or amorphous structure).

FIG. 1 shows an exemplary synthetic strategy for an iono-elastomer using concentrated solutions of end-functionalized PEO106-PPO70-PEO106 triblock copolymer, an amphiphilic polymer, in ethylammonium nitrate (a conductive protic ionic liquid). The two-step synthesis includes dissolving Pluronic® F127 diacrylate (F127-DA) into partially deuterated protic ethylammonium nitrate (dEAN), with a polymer composition of F127-DA of approximately from 20 wt % to 30 wt %. In this example, the F127-DA is present in an amount of 24 wt %. F127-DA is prepared by end-functionalizing the commercial Pluronic® F127 to result in a triblock copolymer with a center polypropylene oxide (PPO) block and two end polyethylene oxide (PEO) blocks. The end-functionalization consists of acrylation of the two hydroxyl end groups, which yields double bonds at the ends of the polymer. The acrylation allows for cross-linking. The solution is then heated to 40° C. and cross-linked using an ultraviolet (UV) light source and a photoinitiator.

The end-functionalized PEO106-PPO70-PEO106 triblock copolymers in ethylammonium nitrate self-assemble into hierarchical face-centered cubic (FCC) micellar crystals, and the micelle coronas cross-link at the end-functionalized PEO blocks (acrylate groups) to generate cross-linked elastomeric ion gels (iono-elastomers). Iono-elastomers can be distinguished from standard ion gels, which flow under deformation and are not highly stretchable. The iono-elastomers, in contrast, exhibit an unprecedented combination of high stretchability, resistance to flow under physical deformation, maintenance of ionic conductivity during stretching, and unique mechano-electrical response. The mechano-electrical response consists of a remarkable and counterintuitive decrease in electrical resistance with strain during uniaxial extension of the iono-elastomer, which is reversible upon load release. Based on in-situ SANS measurements of reversible crystal structure transformations during deformation, the inventors postulate, without being bound by any particular theory, that the origin of the conductivity increase is a reversible formation of ion nano-channels due to a novel microstructural rearrangement specific to the iono-elastomers.

The amphiphilic block-copolymer F127-DA exhibited in FIG. 1 forms spherical micelles in dEAN with the solvophobic PPO center block segregated in the micelle core and the solvophilic PEO end blocks forming the corona, which is solvated by dEAN. At the composition (24 wt %) and temperature (40° C.) exemplified here, the micelles self-assemble into a hierarchical face-centered cubic (FCC) lattice. Upon photo-curing, the lattice structure is unaltered, as indicated by the nearly identical small-angle neutron scattering (SANS) profiles of the solution before and after crosslinking (inset in FIG. 1). The solid lines in the inset of FIG. 1 are best fits to a FCC lattice model with paracrystalline distortion, for which the fitting parameters are the micelle radius R, and the nearest neighbor distance D. The fitting yields R=4.8 nm and D=28 nm for the solution before cross-linking, and R=4.6 nm and D=28 nm after cross-linking. The closeness of the fitted parameters and the close fitting with the FCC lattice model for both sets of SANS data confirm that the hierarchical structure is not modified by the cross-linking reaction.

Even though the structure remains unchanged after cross-linking, the mechanical response of the exemplary F127-DA/dEAN solution is greatly modified. The uncross-linked solution behaves as a “hard gel”, characterized by a very long relaxation time and a pronounced shear-thinning under dynamic and steady flow. Due to the presence of the ionic liquid in the gels and their concomitant high conductivity, this example belongs to the broad category of “ion gels”. After cross-linking, however, the solution loses its fluidity and becomes a soft solid with remarkable flexibility and stretchability. The cross-linked solutions are best described as soft elastomers, and, due to their high ionic conductivity, and to further distinguish them from the (uncross-linked) ion gels, these materials can be termed iono-elastomers.

Other amphiphilic polymers and copolymers and conductive liquids can be used to produce iono-elastomers in addition to F127-DA/dEAN. For example, Pluronic® L121 may be end-functionalized to produce a triblock copolymer capable of self-assembly into micelles and cross-linking at the end functionalizations. Other suitable polymers include functionalized linear polymers, diblock copolymers, and penta-block copolymers. There may also be additive single block polymers such as PEO diacrylate to aid in crosslinking and bridge crosslinking. Each iono-elastomer material may also be composed of different amphiphilic functionalized polymers; the iono-elastomer need not be composed of a single type of amphiphilic functionalized polymer. Blends of polymers of different molecular weights and block composition can be used to create hierarchically organized structures. Depending on the polymers or block copolymers and electrically conductive liquid solvent used, acceptable ranges of concentration of wt % of the polymers/block copolymers that will achieve the hierarchical self-assembly vary.

Moreover, suitable conductive liquids are not limited to dEAN or other ionic liquids. For example, the conductive liquid may be simply water with dissolved salts, acids, bases, or other ionic species to impart conductivity. The conductive liquid solvent may also include propylene carbonate, a protic liquid, a protic ionic liquid, an aprotic ionic liquid, or mixtures thereof. The conductive liquid must merely be capable of inducing the amphiphilic polymers to self-assemble into micelles. Preferred liquids are ionic liquids, which do not evaporate and are thermally stable. Even more preferred are protic ionic liquids.

The micelles may self-organize to form various types of hierarchical gel-like structures, including crystalline or amorphous hierarchical arrangements. The crystalline structures may include, for example, randomly close-packed structures, hexagonally closed-packed structures, and body-centered cubic structures.

Crosslinking may be initiated in one or more ways and does not require both the addition of heat and photoinitiation. For example, any combination of heating, photoinitiation, radiation, ultrasound, or radical polymerization may be used to cross-link the polymer. Crosslinking may also take place at room temperature.

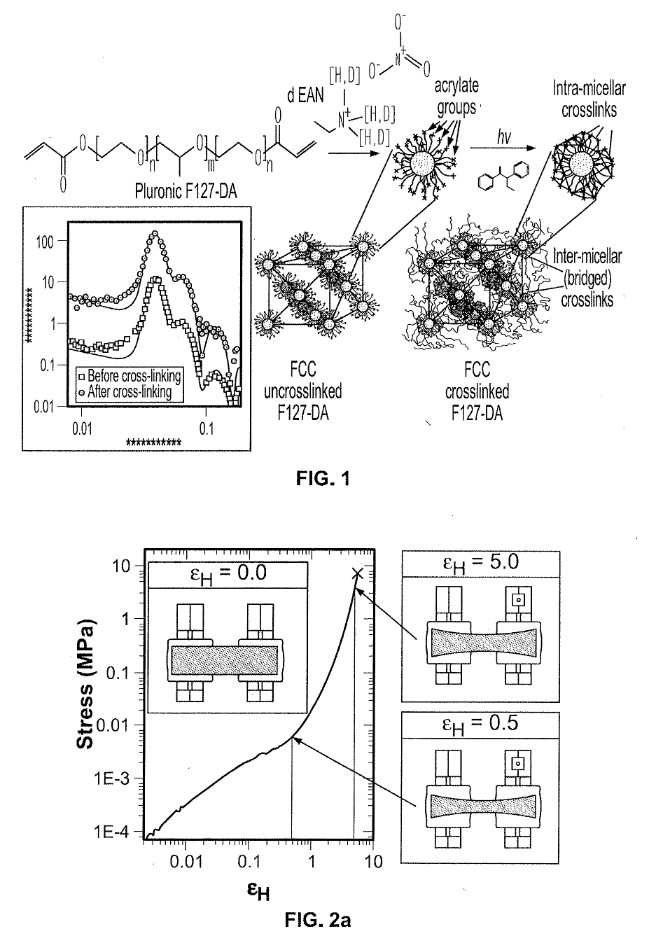

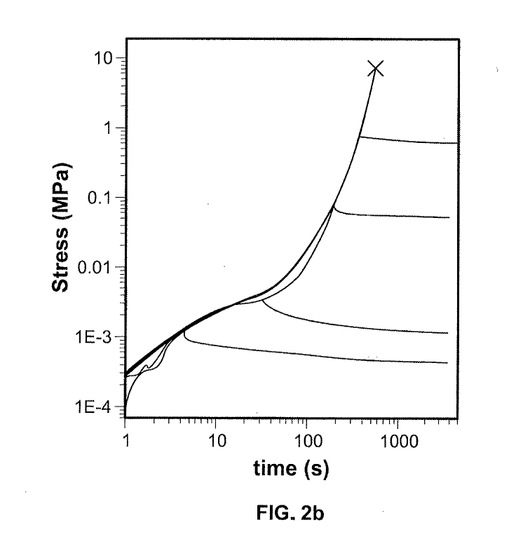

The mechanical response of the exemplary F127-DA/dEAN iono-elastomers was studied under tensile loading using a Sentmanat extensional rheometer (SER). As shown in FIG. 2a, films of the crosslinked samples display elastomeric tensile properties, namely, an initial elastic response (with Young modulus=54 kPa) up to a Hencky strain (ε_{H})˜0.1, followed by yielding and a very pronounced strain-hardening with onset at ε_{H}˜0.5. Hencky strain is defined as ε_{H}=ln(λ)=ln(e+1), where λ and e are the stretch ratio and the engineering extensional strain, respectively. Remarkably large values of both strain to break, ε_{H, strain to break}=4.7±0.8 and tensile strength of 6.9±0.7 MPa are measured. The inventors believe these values are larger than those reported in other hydrogels or crosslinked ion gels. The photographs in FIG. 2a illustrate the high stretchability of iono-elastomers.

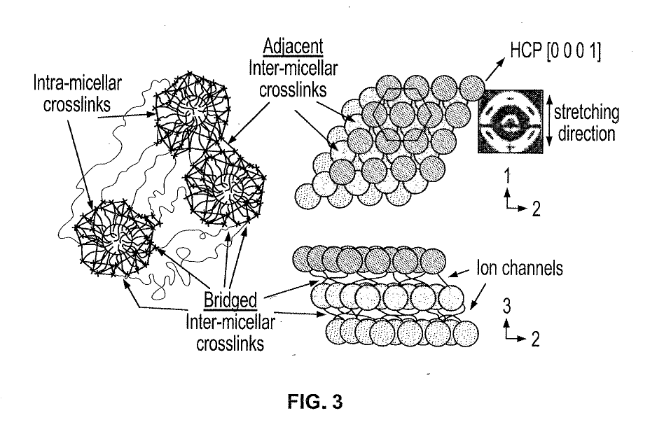

Additionally, iono-elastomer films can support large stresses in comparison to reported ion gels. FIG. 2b shows stress relaxation after stretching to several strain values (ε_{H}=0.032, 0.32, 2 and 4). Larger relaxation is observed in the strain region between the yield and the onset of strain-hardening. Interestingly, the relaxation in the strain-hardening region is negligible. Although the molecular origin of the remarkable tensile behavior of this material is not well understood, and without being bound to any particular theory, the inventors hypothesize that the high stretchability of the iono-elastomers is due to formation of long polymer bridges between micelles created by crosslinking end blocks of copolymer chains in solution with the micelle coronas (see FIG. 1 and FIG. 3) in a crystalline lattice formation, as discussed below. The inventors have observed that the iono-elastomer stretchability properties are benefited by a high concentration of free polymer (i.e., not incorporated into the micelles) contributing to the cross-linked connections between the micelle coronas, primarily in ionic liquids. This hierarchical structure formation is thought to benefit the elasticity of the resulting iono-elastomers without imparting unwanted stiffness. Thus, a preferred embodiment of iono-elastomer results from a high concentration of amphiphilic polymer dissolved in an ionic liquid such that there are large numbers of free amphiphilic polymers in solution, unaffiliated with micelles, which allows exemplary inter- and intra-micellar bridging to occur during cross-linking.

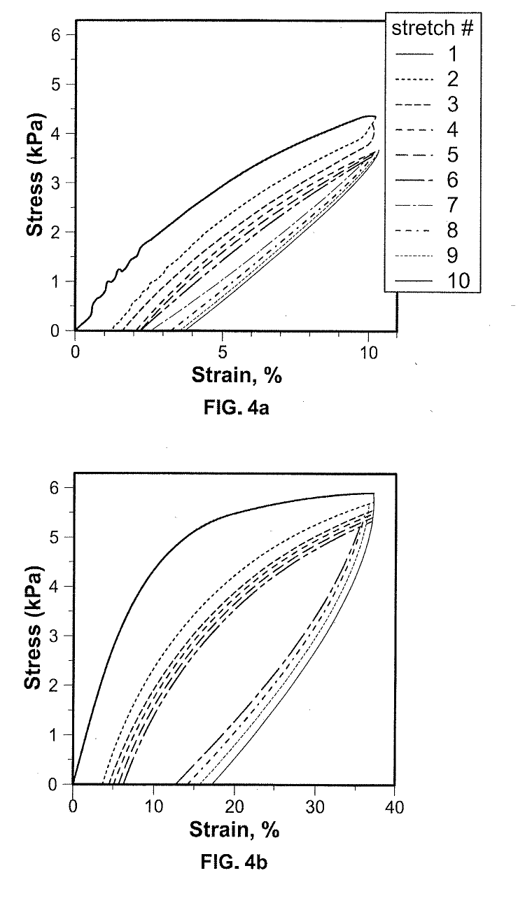

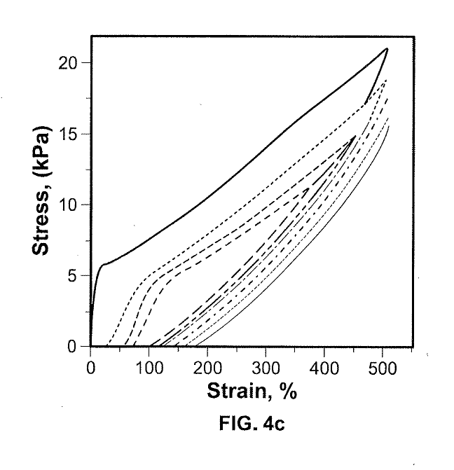

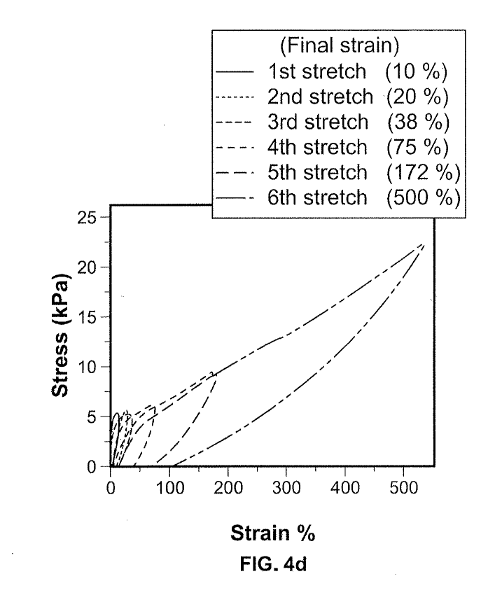

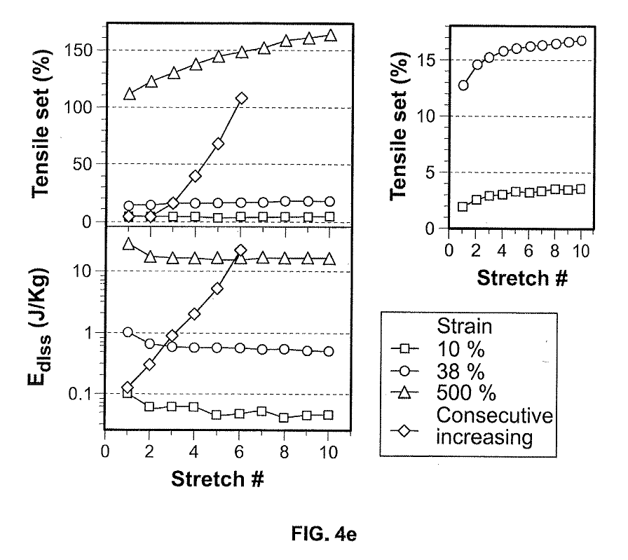

The inventors presume that stretching the sample extends the high entropy random coil configuration of the polymers bridging between the micelles and therefore, the entropic restoring force drives the iono-elastomer to its original configuration after unloading the stress. However, the recovery is not complete and a permanent deformation, known as a “tensile set” is measured after a loading-unloading cycle. Such Irreversibility could be due to pull-out of polymers from the micelles or actual bond breakage. FIGS. 4a-4c shows a series of consecutive loading and unloading cycles for three different maximum strains, which cover the three regimes of deformation; below the yield (strain=10%, FIG. 4a), in the plastic deformation region (strain=38% FIG. 4b), and in the strain-hardening regime (strain=505%, FIG. 4c). Hysteresis is observed in the three regimes, suggesting a certain amount of internal friction and losses due to viscoelasticity. Hysteresis is noticeable larger during the first loading-unloading cycle than during subsequent cycles, which is a typical response of elastomer composites and is known as the Mullins effect. As shown in FIG. 4d, if subsequent cycles are performed with increasing maximum strains, the hysteresis increases accordingly. To quantify the hysteresis, we calculate the dissipated energy during a loading-unloading cycle, E_{diss}, which is the area between the loading and unloading curves. E_{diss} and tensile set are plotted as functions of subsequent stretch cycle number in FIG. 4e. Both the tensile set and the dissipated energy increase by approximately one order of magnitude between each stretching regime studied.

Note that the tensile set continuously increases with subsequent stretch cycles, which indicates fatigue of the material due to microstructural damage. In contrast, E_{diss} does not change after the first stretch cycle, which indicates that molecular friction remains unchanged after multiple cycles.

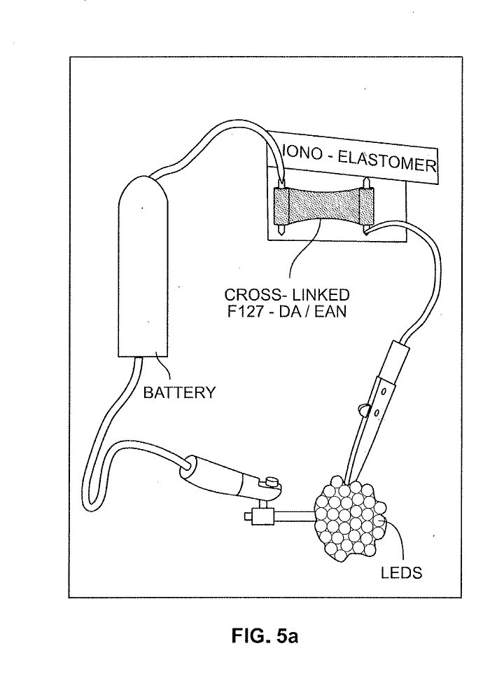

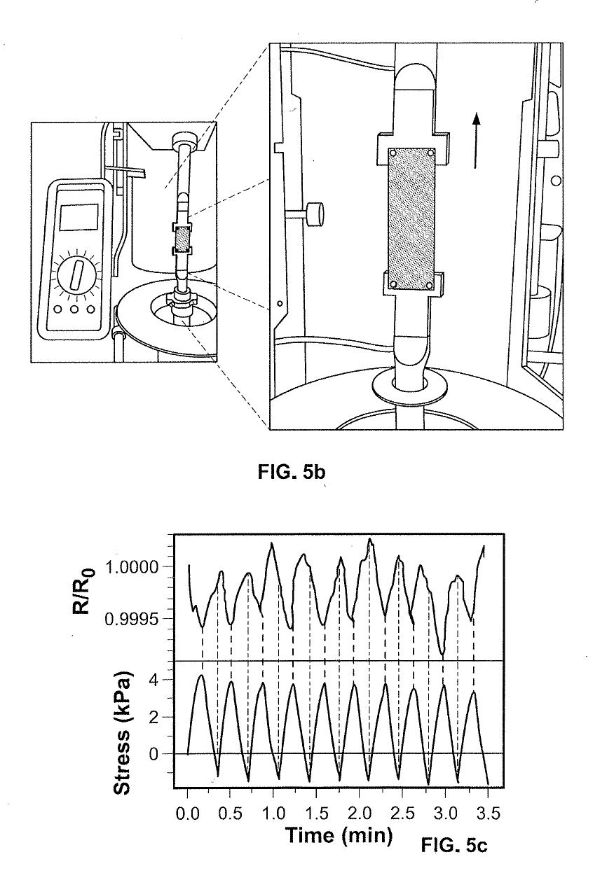

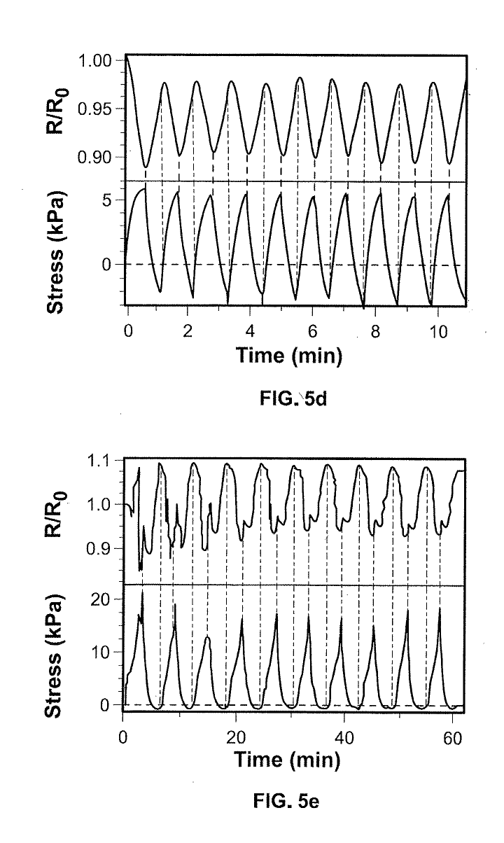

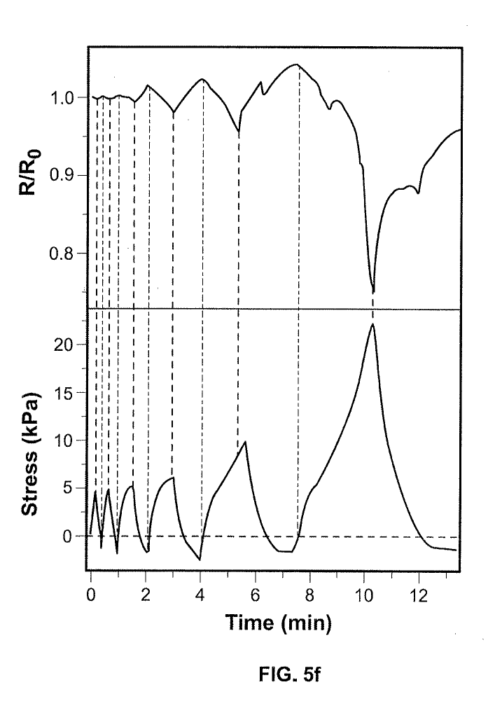

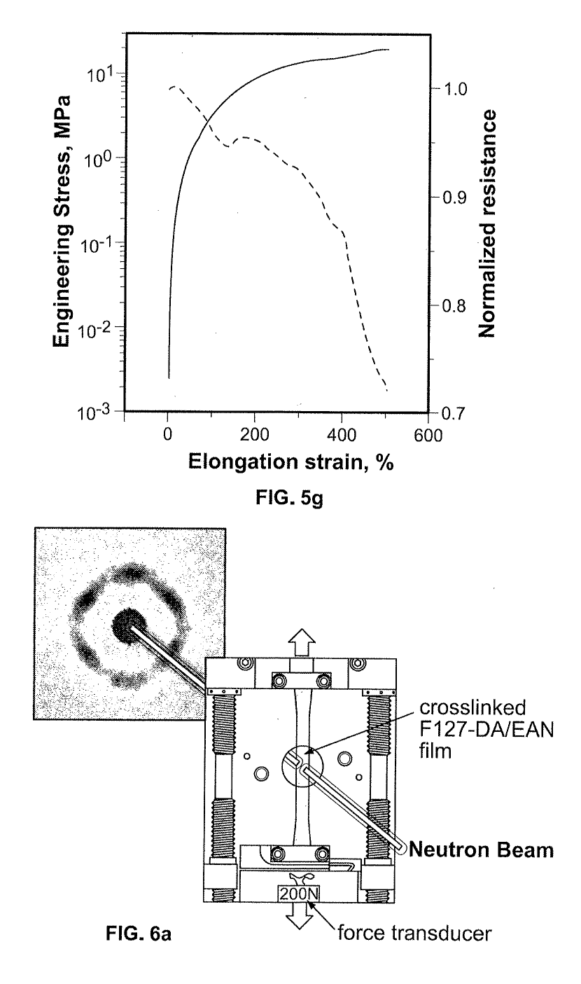

The effect of stretching on the electrical properties is an important property for stretchable electronics. FIGS. 5a-5g present results for the mechano-electrical response of the exemplary F127-DA/dEAN iono-elastomer. First, the sample iono-elastomer was verified as a conductor by connecting a series of LED lights to a battery via a film of the crosslinked sample, as shown in FIG. 5a. The ionic conductivity for the iono-elastomer at 25° C. is 15 mS/cm, which is approximately half that of neat dEAN (28 mS/cm). However, as can be seen in FIG. 5a, the iono-elastomer is a good electrical conductor.

Simultaneous tensile and electrical measurements were performed on a rheometer using a vertical stretching tool where the ends of a film specimen are glued to the upper and lower tools of the instrument and to copper wires that are connected to an ohmmeter, as shown in FIG. 5b. The hysteresis stress data shown in FIGS. 4a-4d are plotted as function of time in FIGS. 5c-5f, respectively. Also plotted in FIGS. 5c-5f is the normalized electrical resistance, R/R₀ (where R₀ is the resistance before stretching), measured with the ohmmeter. Clearly, the change in resistance is in phase with the deformation, and it is proportional to the strain. The latter is more evident in FIG. 5f where strain is increased in subsequent deformation cycles.

Intriguingly, the electrical resistance decreases with strain. This result is counterintuitive given that, for a material with conductivity σ, the resistance is given as

$$R = \frac{1}{σ} \frac{l}{A} \qquad ( Equation 1 )$$

where l is the length of the iono-elastomer sample and A is its cross-sectional area. σ is an intrinsic property, and therefore, it does not change unless the microstructure or chemical composition of the material changes. Stretching the films involves both increase of l and decrease of A, which, according to Equation 1, should result in an increase of the resistance.

The inventors surprisingly observed the opposite outcome in that the resistance decreases with extension as evident in the measurements shown in FIGS. 5a-5f. This counter-intuitive and non-trivial behavior indicates that the intrinsic conductivity of the material changes during the stretching process due to a micro-structural rearrangement. During unloading, the resistance increases nearly to its original value (R₀), which suggests that the microstructure largely recovers its original configuration. Although the reciprocal relation between resistance and strain is observed in all three regimes of deformation, when the stretching surpasses the onset of strain-hardening (strain ˜65%) the resistance recovery occurs in two steps, with an intermediate increase, followed by a second rise such that the resistance reaches values higher than R₀. This reflects a complex series of microstructural transitions occurring internally during the loading-unloading cycles.

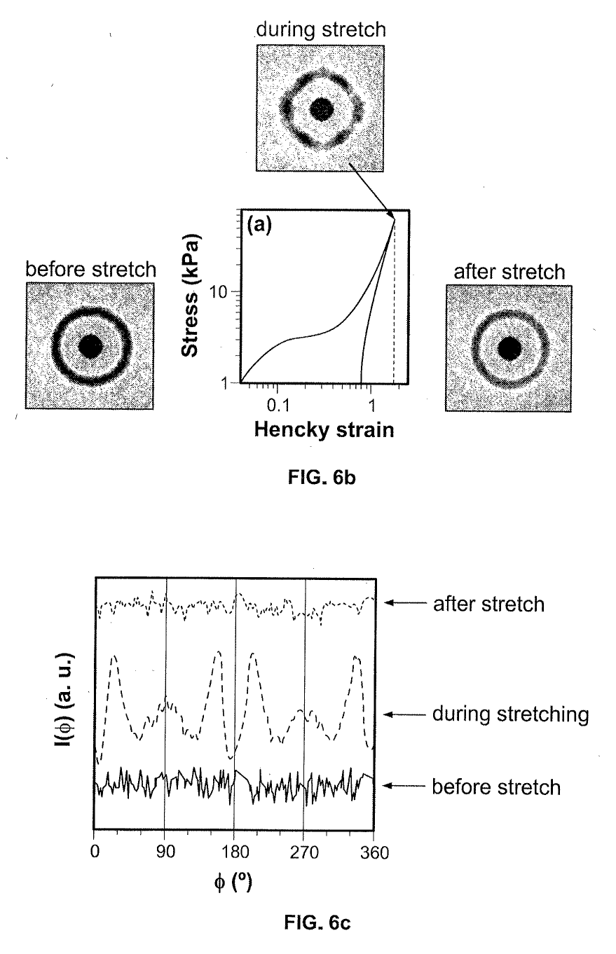

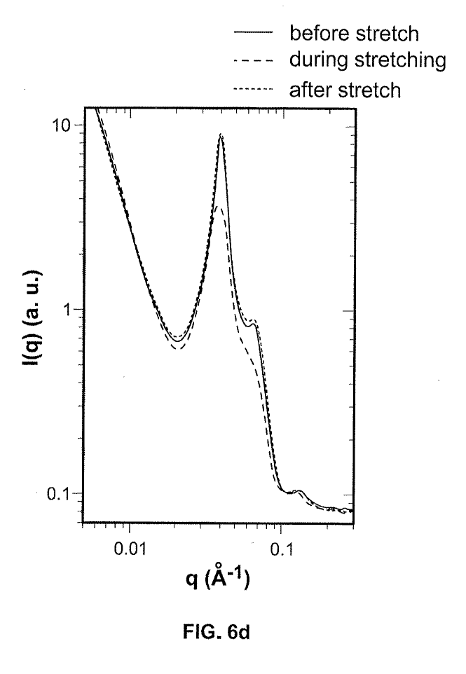

To elucidate the origin of the mechano-electrical response observed in iono-elastomers, in-situ small-angle neutron scattering (SANS) measurements were performed during uniaxial deformation, using a Linkam tensile stage. The neutron beam is focused in the center of the sample to collect SANS data during the deformation (see FIG. 6a). The stress-strain curve measured in the Linkam stage is shown in FIG. 6b, along with the 2D SANS patterns measured at the indicated strain values. Before stretching, an isotropic SANS profile is observed which indicates that the microstructure consists of randomly oriented grains of micelles locally arranged in FCC crystals. Stretching the sample results in the appearance diffraction spots with six-fold symmetry, which indicates the formation of two dimensional hexagonal close-packed (HCP) layers, where the layers are aligned perpendicular to the X-rays beam (i.e., lying in the 1-2 plane of deformation). Although the appearance of the sixfold diffraction pattern in the 2D SANS data is only evident deep into the strain-hardening regime, its formation starts at lower strains, namely, in the plastic deformation regime. This is evident in FIG. 6c, which shows the annular averaged intensity as a function of azimuthal angle, ϕ. The first signs of peak formation can be seen at ε_{H}=0.29, as indicated by the arrows in FIG. 6c. The peak intensity increases with strain, indicating a decrease in the population of randomly oriented FCC grains, which become layered HCP. Remarkably, this transition is reversible upon unloading the stress in the sample, as observed in the 2D SANS pattern labeled as “after stretch” In FIG. 6b and in the corresponding curve in FIG. 6c. Further evidence of this is given in FIG. 6d, which shows azimuthally averaged ID SANS profiles of the sample before, during and after stretching. The SANS profiles before and after stretching are nearly identical, indicating a return to the same micelle configuration.

The inventors believe this is the first report of a FCC to HCP transition driven by uniaxial deformation. On the other hand, shear-induced FCC to HCP transitions have been observed in several colloidal systems in shear flow. Under shear flow, HCP layers are arranged with the layers normal parallel to the velocity gradient direction and the close-packed direction parallel to the velocity direction. The origin of the shear-induced FCC to HCP transition is easy to understand considering that the [111] plane of the FCC lattice has the higher planar density, i.e., adjacent micelles are closer to each other in that plane than in the [100] or [110] planes. The overall higher interaction energy between adjacent micelles in the [111] plane makes it the most resistant to shear stresses. The micelles in the [111] FCC plane have the same configuration as in the [0001] plane of HCP, which has the hexagonal geometry responsible for the six-fold scattering profile (see FIG. 3). Therefore the FCC to HCP transition simply consists of shear-aligning layers of the surviving [111] FCC planes. What is not clear is the mechanism for the FCC to HCP transition induced by extensional deformation for the present iono-elastomer system. Without being bound by any particular theory, the Inventors postulate a mechanism that explains the reversible microstructural transition and the mechano-electrical response observed in the iono-elastomer sample.

Due to the higher planar density, micelles are more prone to crosslink with adjacent neighbors in the [111] plane than in the other planes in the FCC lattice (i.e., “adjacent” inter-micellar crosslinks depicted in FIG. 3). Given that the critical micelle concentration of the exemplary F127-DA/dEAN system is >2 wt %, the inventors assumed that a non-negligible amount of F127-DA chains that remain in solution (i.e., outside of micelles) will form covalently-bonded bridges between micelles upon crosslinking. A relatively larger population of these “bridged” inter-micellar cross-links will connect micelles that are relatively far apart, i.e., not in the [111] plane. Tensile stress applied to this morphology will result in elongation of the bridges (which are initially coiled) and rearrangement of the FCC lattice grains into a lower energy configuration, namely, HCP layers. Therefore, slip occurs between [111] FCC planes that are reinforced by the “adjacent” inter-micellar cross-links. This slip leads to minimal distortion of the inter-micellar hexagonal arrangement, as evidenced by SANS profiles (FIGS. 6a-6c).

The formation of HCP layers perpendicular to the 1-2 plane of deformation produces ion channels between layers, as illustrated in FIG. 3. This configuration results in reduction in tortuosity to ion transport in the stretching direction (1) as compared to the initial configuration of randomly oriented FCC grains. Thus, this microstructural transition evident in the SANS experiments under uniaxial deformation explains the measured decrease in electrical resistance upon stretching (FIGS. 5a-5f). Furthermore, during stretching, mechanical energy is stored in the elongated bridges, which remain connected to the same micelles that were originally in a different configuration. Therefore, these bridges act as a microstructure-memory device, namely, the bridges return to their initial random coil conformation when the stress is released, such that the micelles are pulled back into their original positions. This explains the reversibility of the FCC to HCP transition observed by SANS (FIGS. 6a-6c). This also explains the increase in electric resistance upon unloading due to the increase in tortuosity when the randomly oriented FCC grain morphology is recovered. Clearly, not all the crosslink points survive the stretching, as reflected by the permanent set measured (FIGS. 4a-4e). Further research is warranted to fully understand the mechanism of layering and micelle orientation. For instance, it is not clear why the layer stacking direction is perpendicular to the 1-2 plane of deformation and why the micelles align in the transverse (2) direction.

In summary, the inventors have developed a facile method of preparing ultra-stretchable soft iono-elastomers by sequential self-assembly and chemical cross-linking of a micellar ion gel. These materials provide a combination of high conductivity, remarkable stretchability, tensile properties and mechano-electrical response. Using in-situ SANS measurements, the microstructural origin of the mechano-electrical response was elucidated, namely, the inventors discovered an unprecedented structural phenomenon: a reversible extensional strain-induced FCC to HCP transition. Without being bound by any particular theory, the inventors hypothesize that this transition is responsible for the resistance decrease during stretching, and further, that its reversibility is due to a complex network structure formed during cross-linking of the micellar FCC lattice.

### Devices Incorporating Iono-Elastomers

Wearable electronics, devices, and sensors to be incorporated into clothing, uniforms, and sporting equipment require flexible, robust soft materials with tunable conductivity and high extensibility—so called “stretchable electronics”. The novel mechano-electrical response exhibited by the iono-elastomers described herein may be particularly useful for a range of motion-sensor applications requiring stretchable conductors. Applications include stretchable batteries, wearable sensors and integrated circuits.

Fabrication of such motion-sensing and stretchable electronic materials usually requires the use of hybrid technology that mixes metals, polymers and conductive materials, to tie the system together electronically. Current strategies include composite materials with “wavy” or fractal designs, micro- or nano-structured metal embedded in elastomeric matrices, biphasic solid-liquid metal thin films embedded in elastomeric substrates, composite films of carbon nanotubes or graphene and metals and polymeric materials doped with dispersions of carbon nanotubes in ionic liquids (bucky gels) which are subsequently coated with elastomeric substrates. Manufacturing of these materials involve sophisticated and complex integration of elastomeric substrates with micro- or nano-structured organic or inorganic electronic materials via multi-step and often costly processes.

In contrast, the inventors herein demonstrate a simplified manufacturing process to create stretchable conductor materials applicable for stretchable electronic technologies by self-assembly of concentrated solutions of end-functionalized commercially available, inexpensive amphiphilic polymers into micelles in an electrically conductive liquid, followed by micelle corona cross-linking to generate elastomeric ion gels (termed “iono-elastomers”). The resulting materials exhibit an unprecedented combination of high stretchability (elongation at break is 3000% and tensile strength at break is 200 MPa), tunable ionic conductivity and a unique mechano-electrical response in which electrical resistance decreases with mechanical extension.

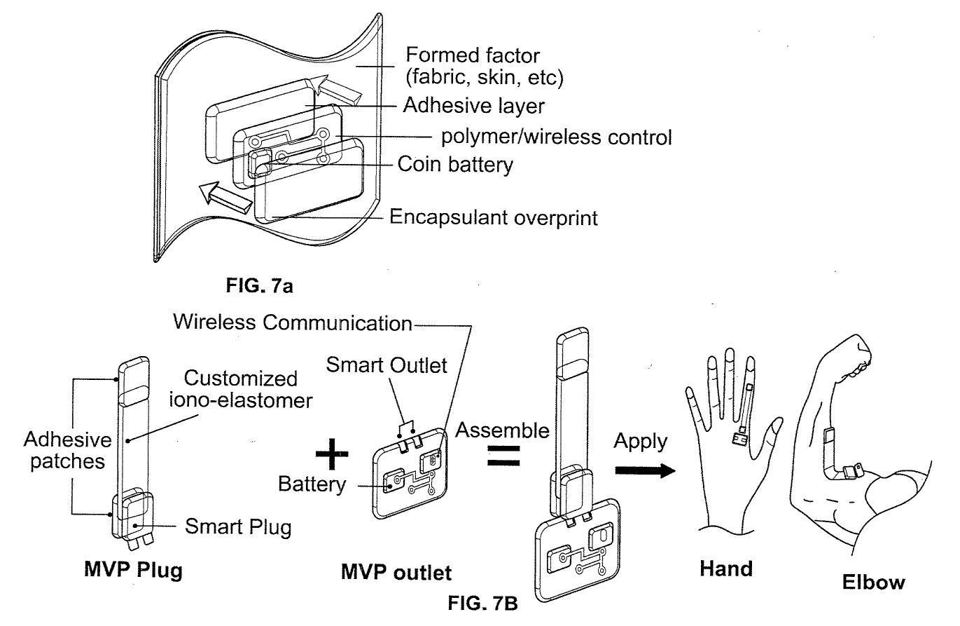

The inventors have adapted the stretchable conductive iono-elastomers for incorporation into clothing, uniforms, or sporting equipment to measure local displacement of muscle under workload, for use in athletic injury prevention, performance analysis, and muscle recovery tracking. As shown in FIG. 7a, an exemplary device incorporating the iono-elastomers is a transparent sensor patch composed of three layered structures. The top layer is an encapsulant overprint, which gives the imparts waterproofing to the device and the iono-elastomer incorporated therein. The middle layer is a stretchable conductive material (i.e., iono-elastomer). The iono-elastomer may be embedded with a wireless system (e.g., Bluetooth) and a battery, which provides the capability of transmitting data from the sensor patch to an external device such as a desktop computer, tablet or phone. The bottom layer is an adhesive layer allowing the exemplary device to directly attach to skin.

In FIG. 7b, another exemplary depiction of a device incorporating the conductive iono-elastomer material is shown. A patch or plug of the iono-elastomer material is coupled with a transmission outlet, such as a wireless communication system. The wireless communication system may be, for example, Bluetooth. The iono-elastomer patch/plug may be plugged into the transmission outlet to form the overall device, which can be attached to different areas of a subject's body to assess muscle exertion and strain.

After attaching the sensor patch to an individual's muscle, the sensor patch can measure the muscle strain under workload and send the strain signal to an external device (desktop computer, tablet, or phone). The strain performance data can be further analyzed via an application associated with the external device to give real-time feedback to interested individuals (e.g., pro-athletes and coaches), giving these interested individuals the ability to customize programs to prevent injury and provide smarter training options.

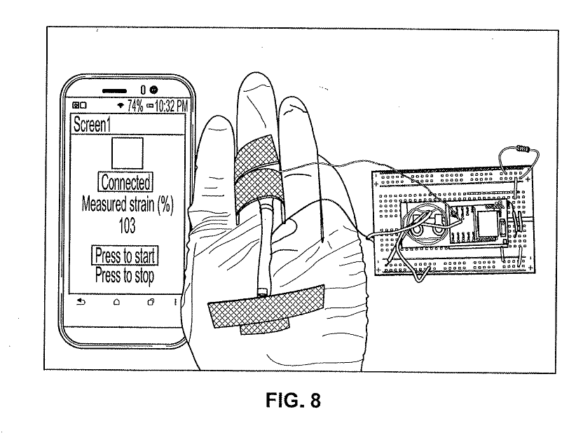

The stretchable iono-elastomers may also be incorporated into other devices and/or sensors capable of being worn on the body and in contact with the skin. For example, the iono-elastomers may be incorporated into clothing (e.g, shirts, pants, socks, footwear, gloves) such that the iono-elastomers are held against the skin to take strain measurements of the muscles beneath. An exemplary embodiment is a glove incorporating the iono-elastomers (see FIG. 8). The iono-elastomers may also be included in bodily accessories, such as watches or wristbands.

### EXAMPLES

The following examples are included to demonstrate the overall nature of the present invention.

### Example 1—Preparation of F12 7-DA/dEAN Solution and Crosslinking

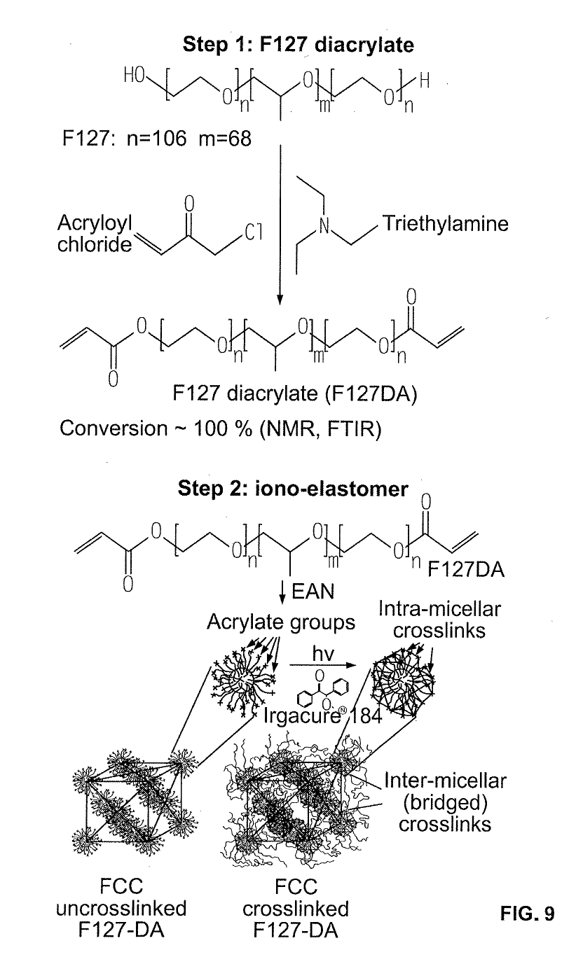

The F127-DA was synthesized following standard procedures (see FIG. 9). Acrylation of the hydroxyl end groups of the commercial triblock copolymer Pluronic® F127 (Aldrich) was performed using trimethylamine and acryloyl chloride in dichloromethane. Partially deuterated ethylammonium nitrate (dEAN) was prepared by three cycles of mixing ethylammonium nitrate (Iolitec) with D₂O, heating to 40° C. with stirring, and drying under vacuum. A 24 wt % F127-DA/dEAN solution with 1 wt % (w.r.t. the polymer weight) of the photoinitiator 1-hydroxycyclohexyl phenyl ketone (Aldrich) was prepared by multiple cycles of mechanical mixing (using a vortex mixer) and centrifugation until obtaining a clear, transparent solution. The solutions were casted on glass slides with rectangular edges to produce films of 10 mm wide and 1 mm thick. After casting, the solutions were cross-linked by exposing them to an UV light source (365 nm, 9.6 mW/cm²) for 30 min. The cross-linked films were carefully peeled off from the glass slides and used for the tensile measurements.

### Example 2—Tensile Measurements and In-Situ SANS Measurements

Three instruments were used for the uniaxial extension measurements of the cross-linked films. A Sentmanat extensional rheometer (SER) was mounted on a strain-controlled ARES-G2 rheometer (TA Instruments) and used to measure the tensile properties of the films up to the break point. A double-sided masking tape was put on the drums to ensure good adhesion of the films. To completely avoid slip on the drums, a UV-curing adhesive (Loctite 352™) was applied to cover both the sample strip and the tape, and subsequently cured with a UV lamp. After curing, the adhesive forms a strong bond between the sample and the tape that remains intact after tensile test. The strips were uniaxially stretched at room temperature, using a constant Hencky strain rate=0.01 s″¹.

Electro-mechanical hysteresis tests were performed in the ARES-G2 rheometer using a vertical stretching tool. The sample was electrically insulated from the rheometer by using double sided tape between the sample and the tools. The ends of the sample are connected to an Ohmmeter (Fluke 289 True-RMS) via copper wires. To avoid slip, the sample and the wires are glued to the tape with a UV-curing adhesive. The electrical resistance was measured and recorded in the Ohmmeter during the deformation. Uniaxial extension was applied to the samples using a constant deformation rate of 0.1 mm/s, in both loading and unloading directions. SANS measurements were carried out at the beam line 12-ID-B at the Advanced Photon Source (APS) at Argonne National Laboratory. The incident neutron energy was 12 keV. A Linkam tensile stage (TST350) was used to apply uniaxial deformation to the sample with a constant rate of 0.1 mm/s while measuring SANS from the sample.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. An electrically conductive iono-elastomer material comprising:
an electrically conductive liquid solvent, and
cross-linked amphiphilic polymer subunit molecules arranged in micelles, the micelles arranged to form a hierarchical gel-like material within the electrically conductive liquid solvent, wherein:
the amphiphilic polymer subunit molecules have end-functionalizations and self-assemble into micelles within the electrically conductive liquid solvent prior to cross-linking,
the end-functionalizations form a corona for each micelle prior to cross-linking, and
the cross-linking occurs between the corona for each micelle and at least one of the end-functionalizations of at least one of another corona or end-functionalizations of free amphiphilic polymer subunit molecules in the electrically conductive liquid solvent.

2. The electrically conductive iono-elastomer material of claim 1, wherein the electrically conductive liquid solvent is at least one of:
water with added salts, acids, bases, or ionic species,
propylene carbonate,
a protic liquid,
a protic ionic liquid,
an aprotic ionic liquid, or
mixtures thereof.

3. The electrically conductive iono-elastomer material of claim 2, wherein the electrically conductive liquid solvent is water with added salts or ionic species.

4. The electrically conductive iono-elastomer material of claim 2, wherein the electrically conductive liquid solvent is a protic ionic liquid.

5. The electrically conductive iono-elastomer material of claim 4, wherein the protic ionic liquid is ethylammonium nitrate.

6. The electrically conductive iono-elastomer material of claim 5, wherein the ethylammonium nitrate is at least partially deuterated.

7. The electrically conductive iono-elastomer material of claim 1, wherein the amphiphilic polymer subunit molecules include at least one of single block polymers, di-block copolymers, tri-block copolymers, penta-block copolymers, functionalized linear polymers, or mixtures thereof.

8. The electrically conductive iono-elastomer material of claim 7, wherein the amphiphilic polymer subunit molecules include tri-block copolymers.

9. The electrically conductive iono-elastomer material of claim 8, wherein the tri-block copolymers include end-functionalized polyethylene oxide-polypropylene oxide-polyethylene oxide triblock copolymer.

10. The electrically conductive iono-elastomer material of claim 1, wherein the amphiphilic polymer subunit molecules are present in an amount of between about 5 wt % and 60 wt %.

11. The electrically conductive iono-elastomer material of claim 1, wherein the hierarchical gel-like material comprises face-centered cubic structures.

12. The electrically conductive iono-elastomer material of claim 1, wherein the hierarchical gel-like material comprises body-centered cubic structures.

13. The electrically conductive iono-elastomer material of claim 1, wherein the hierarchical gel-like material comprises hexagonally close-packed or randomly close-packed structures.

14. The electrically conductive iono-elastomer material of claim 1, wherein the hierarchical gel-like material is amorphous.

15. The electrically conductive iono-elastomer material of claim 1, further comprising a plurality of cross-linked solvophilic polymer subunit molecules, wherein the solvophilic polymer subunit molecules have end-functionalizations and the cross-linking occurs between the corona for each micelle and at least one of the plurality of solvophilic polymer subunit molecules in the electrically conductive liquid solvent.

16. A method for producing the electrically conductive iono-elastomer material of claim 1, the method comprising:
dissolving a plurality of the amphiphilic polymer subunit molecules having end-functionalizations in the electrically conductive liquid solvent such that the plurality of the amphiphilic polymer subunit molecules form micelles, wherein:
the end-functionalizations of the amphiphilic polymer subunit molecules in the micelles form micellar coronas, and
the micelles self-organize into hierarchical gel-like structures; and
cross-linking the micellar coronas with at least one of the end-functionalizations of at least one of another corona or end-functionalizations of free amphiphilic polymer subunit molecules in the electrically conductive liquid solvent.

17. The method of claim 16, wherein the cross-linking step is initiated at room temperature.

18. The method of claim 16, wherein the cross-linking step is initiated with at least one of increase in heat from room temperature, with photoinitiators, with radiation, with ultrasound, or with radical polymerization.

19. The method of claim 18, wherein the cross-linking step is initiated at least partially with photoinitiators and UV light.

20. The method of claim 16, wherein the electrically conductive liquid solvent is at least one of:
water with added salts, acids, bases, or ionic species,
propylene carbonate,
a protic liquid,
a protic ionic liquid,
an aprotic ionic liquid, or
mixtures thereof.

21. The method of claim 20, wherein the electrically conductive liquid solvent is water with added salts or ionic species.

22. The method of claim 20, wherein the electrically conductive liquid solvent is a protic ionic liquid.

23. The method of claim 22, wherein the protic ionic liquid is ethylammonium nitrate.

24. The method of claim 23, wherein the ethylammonium nitrate is at least partially deuterated.

25. The method of claim 16, wherein the plurality of the amphiphilic polymer subunit molecules include at least one of single block polymers, di-block copolymers, tri-block copolymers, penta-block copolymers, functionalized linear polymers, or mixtures thereof.

26. The method of claim 25, wherein the plurality of the amphiphilic polymer subunit molecules includes tri-block copolymers.

27. The method of claim 26, wherein the tri-block copolymers include polyethylene oxide-polypropylene oxide-polyethylene oxide triblock copolymer.

28. The method of claim 16, wherein the plurality of the amphiphilic polymer subunit molecules is present in an amount of between about 5 wt % and 60 wt %.

29. The method of claim 16, wherein the hierarchical gel-like material comprises a face-centered cubic structure.

30. An electronic device configured to sense motion and stress of muscles, the electronic device comprising:
the electrically conductive iono-elastomer material of claim 1; and
at least one of:
an encapsulant layer for covering the iono-elastomer material, or
a layer of adhesive configured to attach the electronic device to skin, wherein
the electronic device is configured to be placed on at least one muscle to collect data on at least one of motion or muscle strain on the at least one muscle.

31. The electronic device of claim 30, wherein the device includes the encapsulant layer and the layer of adhesive, and the electrically conductive iono-elastomer material comprises a layer between the encapsulant layer and the layer of adhesive.

32. The electronic device of claim 30, wherein the electronic device is transparent.

33. The electronic device of claim 30, wherein the electronically conductive iono-elastomer material is embedded with a wireless system.

34. The electronic device of claim 33, wherein the electronic device is in wireless communication with an external device such that the collected data on at least one of motion or muscle strain is sent to the external device.

35. The electronic device of claim 34, wherein the external device is at least one of a computer, a tablet, or a phone.

36. The electronic device of claim 35, wherein the external device includes an application configured to analyze the collected data.

37. The electronic device of claim 33, wherein the electronically conductive iono-elastomer material is embedded with a battery.

38. A wearable device configured to sense motion and stress of muscles, the wearable device comprising:
an article of clothing or a wearable accessory; and
the electrically conductive iono-elastomer material of claim 1.

39. The wearable device of claim 38, wherein the wearable device is a glove.
